(19) United States
(12) Patent Application Publication
(10) Pub. No.: US 2019/0189270 A1
(43) Pub. Date: Jun. 20, 2019

(54) HOSPITAL INFORMATION APPARATUS, HOSPITAL INFORMATION SYSTEM, AND HOSPITAL INFORMATION PROCESSING METHOD

(71) Applicant: Canon Medical Systems Corporation, Otawara-shi (JP)

(72) Inventors: Hideaki ISHII, Nasushiobara (JP); Satoshi Wakai, Nasushiobara (JP)

(73) Assignee: Canon Medical Systems Corporation, Otawara-shi (JP)

(21) Appl. No.: 16/217,334

(22) Filed: Dec. 12, 2018

(30) Foreign Application Priority Data
Dec. 14, 2017 (JP) 2017-239674

(51) Int. Cl. G16H 40/20; G16H 30/20; G16H 50/30

(52) U.S. Cl. CPC G16H 40/20; G16H 50/30; G16H 30/20

(57) ABSTRACT

A hospital information apparatus according to an embodiment includes a processing circuit. The processing circuit is configured to obtain past information related to a subject for whom an examination order is issued. The processing circuit is configured to set one or more additional examination orders on the basis of an analysis performed on the past information. The processing circuit is configured to determine a proposed order from among the additional examination orders on the basis of a relationship between the examination order and the additional examination orders.

### CROSS-REFERENCE TO RELATED APPLICATIONS

This application is based upon and claims the benefit of priority from Japanese Patent Application No. 2017-239674, filed on Dec. 14, 2017; the entire contents of which are incorporated herein by reference.

### FIELD

Embodiments described herein relate generally to a hospital information apparatus, a hospital information system, and a hospital information processing method.

### BACKGROUND

Generally speaking, medical doctors (hereinafter, “doctor”) order medical examinations (hereinafter, “examinations”) to be performed on patients by using an X-ray Computed Tomography (CT) apparatus or the like, while designating a site (e.g., the heart) suspected to have a lesion. According to the orders for the examinations (hereinafter, “examination orders”), an image taking process and a reconstruction process are performed on a region (a region of interest) that covers the designated site (e.g., the heart), so as to check the state of the lesion in the acquired image. However, it is often the case that a lesion is present in another site (e.g., a lung) in a region (a non region of interest) that is different from the region of interest indicated in the designation by the doctor.

### BRIEF DESCRIPTION OF THE DRAWINGS

FIG. 1 is a diagram illustrating an exemplary configuration of a hospital information apparatus and a hospital information system according to a first embodiment;

FIG. 2 is a diagram illustrating an exemplary configuration in which a radiation department information server apparatus is provided with an additional process proposing function;

FIG. 3 is a sequence chart illustrating an example of a process performed when a proposed order suggesting expanding an examination area and an image taking area is presented to a doctor;

FIG. 4A is a drawing illustrating an example of an examination order;

FIG. 4B is a drawing illustrating an example of past information;

FIG. 4C is a drawing illustrating an example of a metastasis possibility judgment table;

FIG. 4D is a drawing illustrating examples of lesion candidates;

FIG. 4E is a drawing illustrating examples of examination area candidates;

FIG. 4F is a drawing illustrating examples of additional examination orders (candidates);

FIG. 4G is a drawing illustrating an example of a site map;

FIG. 4H is a drawing illustrating an example of a proposed order;

FIG. 5 is a sequence chart illustrating an example of a process performed when a proposed order suggesting a reconstruction process is presented to a medical technologist;

FIG. 6A is a drawing illustrating an example of past information;

FIG. 6B is a drawing illustrating examples of additional examination orders (candidates);

FIG. 6C is a drawing illustrating an example of a proposed order;

FIG. 7 is a sequence chart illustrating an example of a process performed when a reconstruction process is automatically performed;

FIG. 8 is a diagram illustrating an exemplary configuration in which a radiation department information server apparatus is provided with an additional process proposing function according to a second embodiment; and

FIG. 9 is a drawing illustrating examples of additional examination orders (candidates).

### DETAILED DESCRIPTION

A hospital information apparatus according to an embodiment includes a processing circuit. The processing circuit is configured to obtain past information related to an examined subject (hereinafter, “patient”) for whom an examination order is issued. The processing circuit is configured to set one or more additional examination orders on the basis of an analysis performed on the past information. The processing circuit is configured to determine a proposed order from among the additional examination orders on the basis of a relationship between the examination order and the additional examination orders.

Exemplary embodiments of a hospital information apparatus, a hospital information system, and a hospital information processing method will be explained below, with reference to the accompanying drawings. Possible embodiments are not limited to the embodiments described below. Further, it is possible, in principle, to similarly apply each of the embodiments and the modification examples to any other embodiment or any other modification example.

### First Embodiment

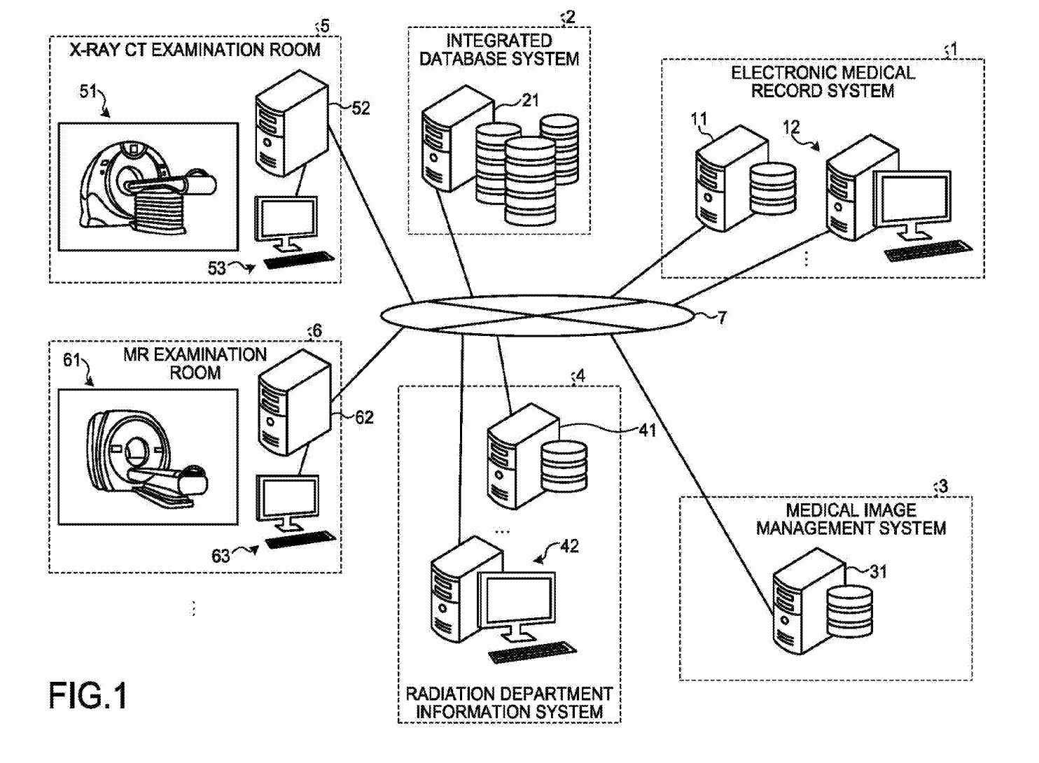

FIG. 1 is a diagram illustrating an exemplary configuration of a hospital information apparatus and a hospital information system according to a first embodiment. FIG. 1 illustrates an electronic medical record system 1, an integrated database system 2, a medical image management system 3, a radiation department information system 4, an X-ray CT examination room 5, and a Magnetic Resonance (MR) examination room 6. The electronic medical record system 1 is an information system configured to manage electronic medical records used for recording details of diagnoses and treatments and includes an electronic medical record server apparatus 11 and at least one electronic medical record terminal device 12. The electronic medical record server apparatus 11 is a computer apparatus configured to perform processes related to the management of the electronic medical records. The electronic medical record server apparatus 11 may be configured by using one or more computer apparatuses depending on the scale of the system. The electronic medical record terminal device 12 is used by doctors, nurses, and the like who input and/or refer to the electronic medical records. One or more electronic medical record terminal devices 12 may be provided depending on the scale of the system. The electronic medical record server apparatus 11 and the electronic medical record terminal device 12 are connected to an intra-hospital network 7 that is either wired or wireless.

The integrated database system 2 is a database system that is not dependent on vendors (suppliers of computer software or hardware) and is configured to store therein data for the electronic medical record system 1, the medical image management system 3, the radiation department information system 4, and the like. The integrated database system 2 includes an integrated database server apparatus 21. The integrated database server apparatus 21 is a computer apparatus configured to perform processes related to managing databases. The integrated database server apparatus 21 is configured by using one or more computer apparatuses depending on the scale of the system. The integrated database server apparatus 21 is connected to the intra-hospital network 7. It is also possible to omit the integrated database system 2. In that situation, the data for the electronic medical record system 1, the medical image management system 3, the radiation department information system 4, and the like is stored in each of the systems.

The medical image management system 3 is an information system configured to manage medical images such as X-ray CT images, MR images, and the like. The medical image management system 3 may be configured to manage not only image data after reconstruction processes hut also data (raw data) before reconstruction processes. The medical image management system 3 includes a medical image management server apparatus 31. The medical image management server apparatus 31 is a computer apparatus configured to perform processes related to the management of the medical images. The medical image management server apparatus 31 may be configured by using one or more computer apparatuses depending on the scale of the system. The medical image management server apparatus 31 is connected to the intra-hospital network 7.

The radiation department information system 4 is an information system configured to manage information in the radiation department of a hospital and includes a radiation department information server apparatus 41 and at least one radiation department information terminal device 42. The radiation department information server apparatus 41 is a computer apparatus configured to perform a process related to the management of the information in the radiation department. The radiation department information server apparatus 41 may be configured by using one or more computer apparatuses depending on the scale of the system. The radiation department information terminal device 42 is used by medical technologists in the radiation department or the like. One or more radiation department information terminal devices 42 may be provided depending on the scale of the system. The radiation department information server apparatus 41 and the radiation department information terminal device 42 are connected to the intra-hospital network 7.

In the X-ray CT examination room 5, an X-ray CT apparatus main body 51, a console device main body 52, and an input/output device 53 are provided. The X-ray CT apparatus main body 51 includes: a gantry device provided with an X-ray tube, an X-ray detector, a rotating frame, and the like; and a couch device on which an examined subject (a patient) is placed. The console device main body 52 is a computer apparatus configured to control operations of the X-ray CT apparatus main body 51 and has connected thereto the input/output device 53 including a monitor and a keyboard, or the like. The console device main body 52 is connected to the intra-hospital network 7.

In the MR examination room 6, an MR apparatus main body 61, a console device main body 62, and an input/output device 63 are provided. The MR apparatus main body 61 includes: a gantry device provided with a magnet, an RF coil, a radio frequency circuit system, a gradient magnetic field system, and the like; and a couch device on which the examined patient (the patient) is placed. The console device main body 62 is a computer apparatus configured to control operations of the MR apparatus main body 61 and has connected thereto the input/output device 63 including a monitor and a keyboard, or the like. The console device main body 62 is connected to the intra-hospital network 7.

In the present example, the X-ray CT apparatus and the MR apparatus are used as examination apparatuses. For each of these types of apparatuses, two or more apparatuses may be provided. Further, one or more apparatuses in any other modality (e.g., ultrasound diagnosis apparatuses) may be provided.

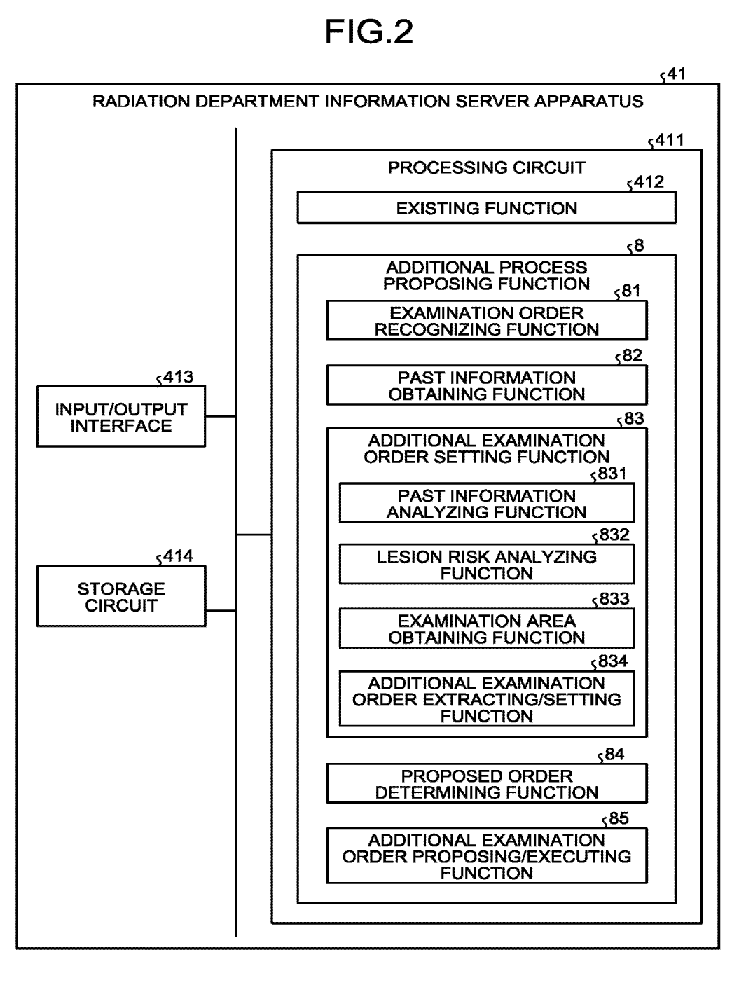

FIG. 2 is a diagram illustrating an exemplary configuration in which the radiation department information server apparatus 41 is provided with an additional process proposing function 8. The additional process proposing function 8 has a function configured either to propose or to automatically execute an additional process, with respect to an examination order issued by a doctor. Instead of being provided for the radiation department information server apparatus 41, the additional process proposing function 8 may be provided for the integrated database server apparatus 21, for the electronic medical record server apparatus 11, or for the medical image management server apparatus 31. Further, the additional process proposing function 8 may be provided for a dedicated server apparatus.

The computer apparatus such as the radiation department information server apparatus 41 provided with the additional process proposing function 8 is an example of the hospital information apparatus. An information system including: the computer apparatus such as the radiation department information server apparatus 41 provided with the additional process proposing function 8; and another computer apparatus configured to exchange information with the computer apparatus through communication is an example of the hospital information system.

In FIG. 2, the radiation department information server apparatus 41 includes a processing circuit 411, an input/output interface 413, and a storage circuit 414. The processing circuit 411, the input/output interface 413, and the storage circuit 414 are connected together via a bus.

The processing circuit 411 is configured to control operations of the entirety of the radiation department information server apparatus 41. For example, the processing circuit 411 is realized by using a processor. For example, in addition to an existing function 412 of the radiation department information server apparatus 41, the processing circuit 411 includes the additional process proposing function 8. The existing function 412 is configured, for example, to input, store, process, and output information within the radiation department. As explained above, the additional process proposing function 8 is configured either to propose or to automatically execute the additional process, with respect to the examination order issued by a doctor. The additional process proposing function 8 includes an examination order recognizing function 81, a past information obtaining function 82, an additional examination order setting function 83, a proposed order determining function 84, and an additional examination order proposing/executing function 85. The additional examination order setting function 83 includes a past information analyzing function 831, a lesion risk analyzing function 832, an examination area obtaining function 833, and an additional examination order extracting/setting function 834.

For example, the processing circuit 411 is configured to perform a process as the radiation department information server apparatus 41 by controlling the radiation department information server apparatus 41, as a result of reading and executing a computer program (hereinafter, “program”) corresponding to the existing function 412 from the storage circuit 414.

Further, for example, the processing circuit 411 is configured either to propose or to automatically execute the additional process with respect to the examination order issued by a doctor, by controlling the radiation department information server apparatus 41, as a result of reading and executing a program orresponding to the additional process proposing function 8 from the storage circuit 414.

The examination order recognizing function 81 included in the additional process proposing function 8 is configured to recognize the examination order issued by the doctor in the electronic medical record system 1. The past information obtaining function 82 is configured to obtain past information related to the patient (the examined subject) for whom the examination order is issued, on the basis of information included in the examination order recognized by the examination order recognizing function 81, such as a patient ID identifying the patient, for example. The past information includes not only general attributes such as the age, the gender, and the like of the patient, but also non-image or image information such as a medical history of the patient (the patient's medical history), medical histories of one or more family members (family medical histories), an imaging history using X-ray CT and the like, a blood test history, and the like.

The additional examination order setting function 83 is configured to set one or more additional examination orders (candidates) for examining the patient for a lesion suspected to occur in the patient, by analyzing the past information obtained by the past information obtaining function 82. Each of the one or more additional examination orders is an order for examining the patient for a lesion that is different from the lesion for which an examination is performed under the examination order. The examination order and the additional examination orders each instruct either that an image of the patient be taken by using a medical image diagnosis apparatus or that a specimen acquired from the patient be analyzed. As a more detailed function, the past information analyzing function 831 included in the additional examination order setting function 83 is configured to analyze the past information obtained by the past information obtaining function 82. For example, the past information analyzing function 831 recognizes items in individual pieces of information included in the past information and extracts information that can be used for speculating lesions (e.g., the patient's medical history, the family medical histories, and the like) and information that can be used for judging about reconstruction processes (e.g., the imaging history or the like).

The lesion risk analyzing function 832 is configured to speculate a lesion having a possibility of occurring in the patient, as a lesion risk analysis, on the basis of the past information analyzed by the past information analyzing function 831. For example, when the patient's medical history indicates that he/she had “cancer” at a site, the lesion risk analyzing function 832 speculates the lesion having a possibility of occurring in the patient, on the basis of a possibility of the cancer metastasizing to another site, a possibility of recurrence, or the like. The examination area obtaining function 833 is configured to obtain an examination area corresponding to the lesion speculated by the lesion risk analyzing function 832. In this situation, the examination area denotes an area of which a diagnosis-purpose image is to be obtained for the purpose of checking for the lesion. When an image is to be newly taken, the examination area denotes an area to be reconstructed reconstruction area) within an area to be imaged (an image taking area). Alternatively, when raw data that has already been taken is subject to the process, the examination area denotes an area to be reconstructed within the area of the raw data. The additional examination order extracting/setting function 834 is configured to set the additional examination orders (candidates), from among candidates obtained by the examination area obtaining function 833 as the examination areas. Further, the additional examination order extracting/setting function 834 is configured to judge whether or not it is possible to substitute all or a part of the examination areas with a reconstructed image of either a part within an image taking area used in the past indicated in the image taking history or a part that is within a new image taking area but is not subject to an examination. When the substitution is possible, the additional examination order extracting/setting function 834 sets an additional examination order (a candidate) indicating the reconstruction process.

The proposed order determining function 84 is configured to determine a proposed order that is either to be presented to a user (e.g., a , a medical technologist, or the like) or to be automatically executed, from among the additional examination orders, on the basis of a relationship between the examination order recognized by the examination order recognizing function 81 and the additional examination orders set by the additional examination order setting function 83. The additional examination order proposing/executing function 85 is configured either to present the proposed order determined by the proposed order determining function 84 to the user (e.g., a doctor, a medical technologist, or the like) or to automatically execute the proposed order.

The processing circuit 411 realizing the past information obtaining function 82 is an example of the obtaining circuit. The processing circuit 411 realizing the additional examination order setting function 83 is an example of the setting circuit. The processing circuit 411 realizing the proposed order determining function 84 is an example of the determining circuit.

The input/output interface 413 is configured to establish a connection with the intra-hospital network 7 and a connection with an input device such as a mouse and/or a keyboard and with an output device such as a display device. The input device is configured to receive various types of input operations from an operator and to convert the received input operations into electrical signals. Examples of the input device include a mouse and a keyboard, as well as a trackball, a switch, a button, a joystick, a touch panel, and the like. The display device is configured to display various types of information. For example, the display device outputs a Graphical User Interface (GUI) or the like used for receiving various types of operations from the operator. The display device may be a liquid crystal display device or a Cathode Ray Tube (CRT) display device.

The storage circuit 414 is realized by using a semiconductor memory element such as a Random Access Memory (RAM) or a flash memory, or a hard disk, an optical disk, or the like. For example, the storage circuit 414 is configured to store therein data used by the radiation department information server apparatus 41 and one or more programs executed by the radiation department information server apparatus 41. The storage circuit 414 may also be used as a non-transient storage medium realized with hardware.

The term “processor” used in the explanations above denotes, for example, a Central Processing Unit (CPU), a Graphics Processing Unit (GPU), or a circuit such as an Application Specific integrated Circuit (ASIC) or a programmable logic device (e.g., a Simple Programmable Logic Device [SPLD], a Complex Programmable Logic Device [CPLD], or a Field Programmable Gate Array [FPGA]). The one or more processors realize the functions thereof by reading and executing corresponding programs stored in the storage circuit 414. in this situation, instead saving the programs in the storage circuit 414, it is also acceptable to directly incorporate the programs in the circuits of the processors. In that situation, the processors realize the functions thereof by reading and executing the programs incorporated in the circuits thereof. Further, the processors in the present embodiments do not each necessarily have to be structured as a single circuit. It is also acceptable to structure one processor by combining together a plurality of independent circuits so as to realize the functions thereof.

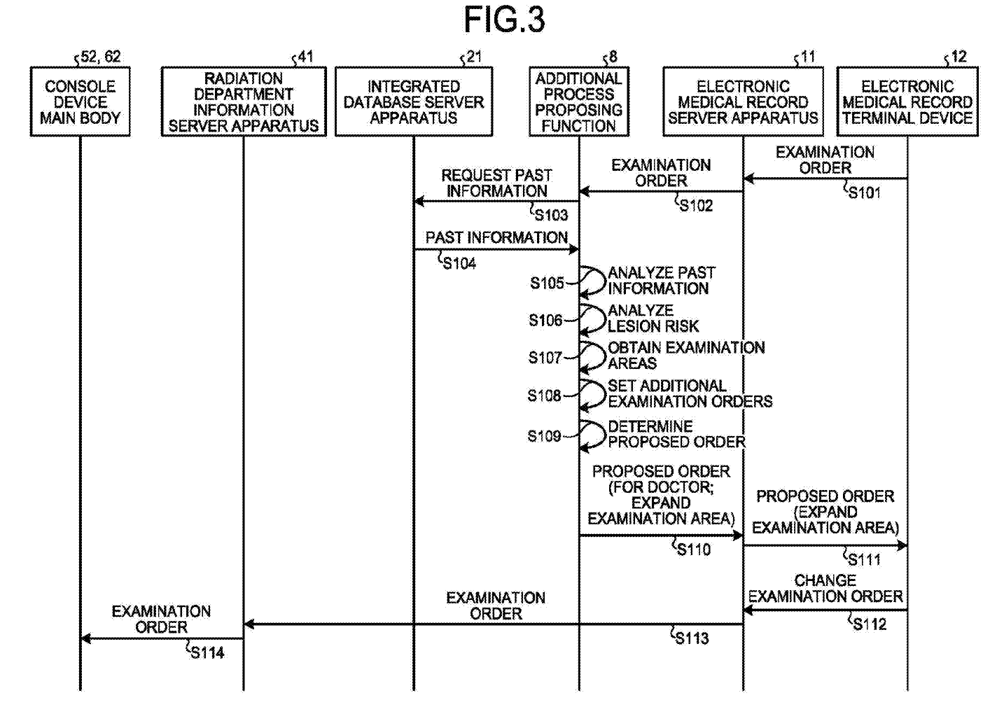

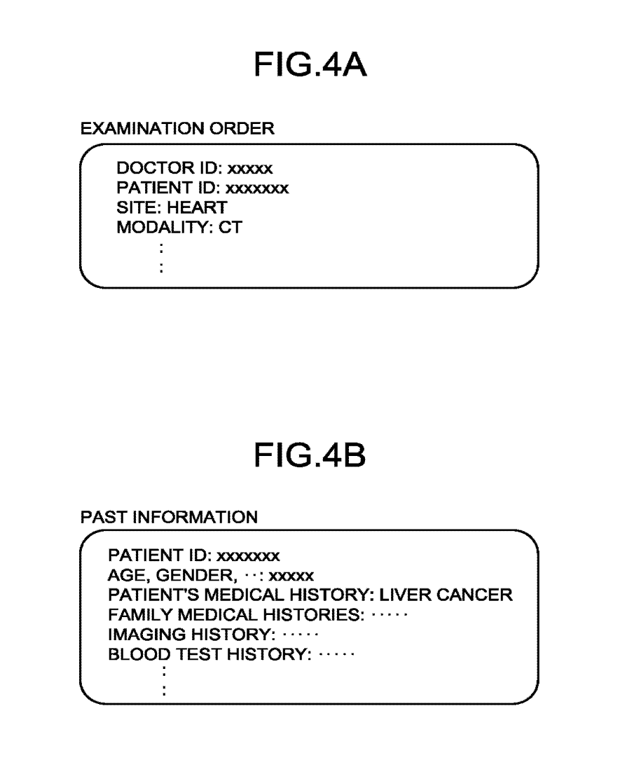

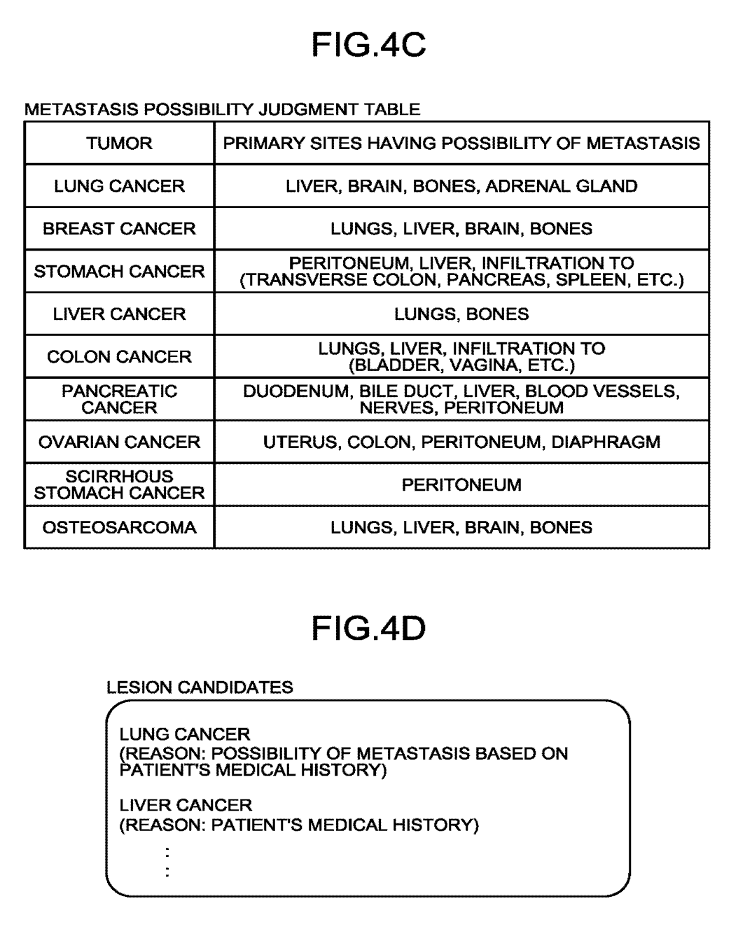

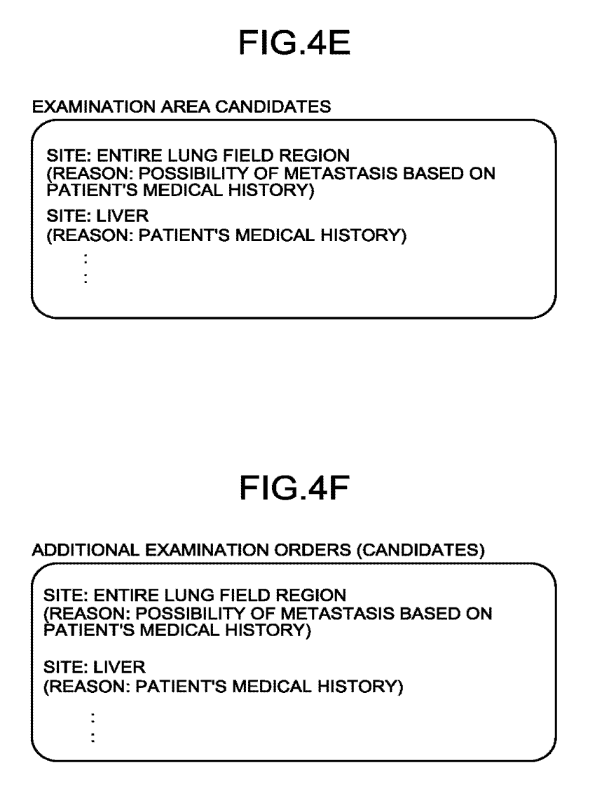

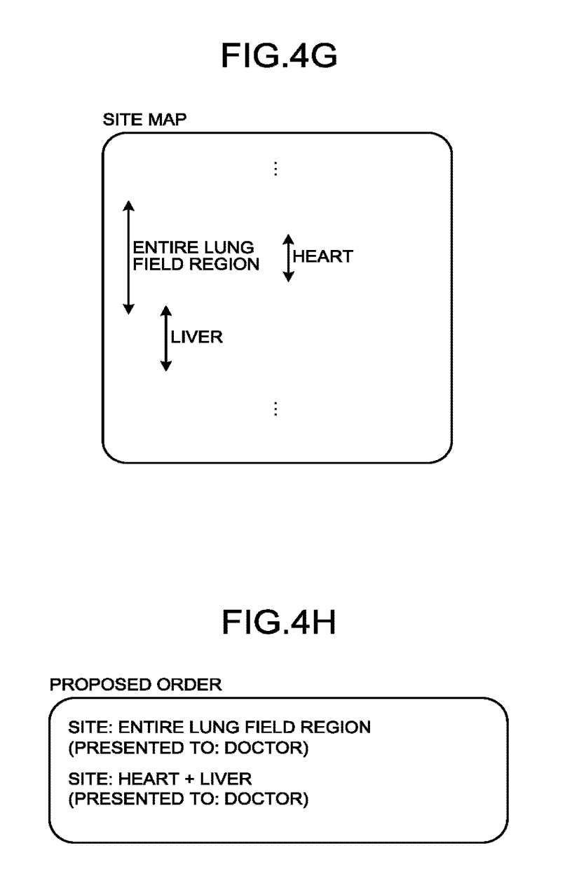

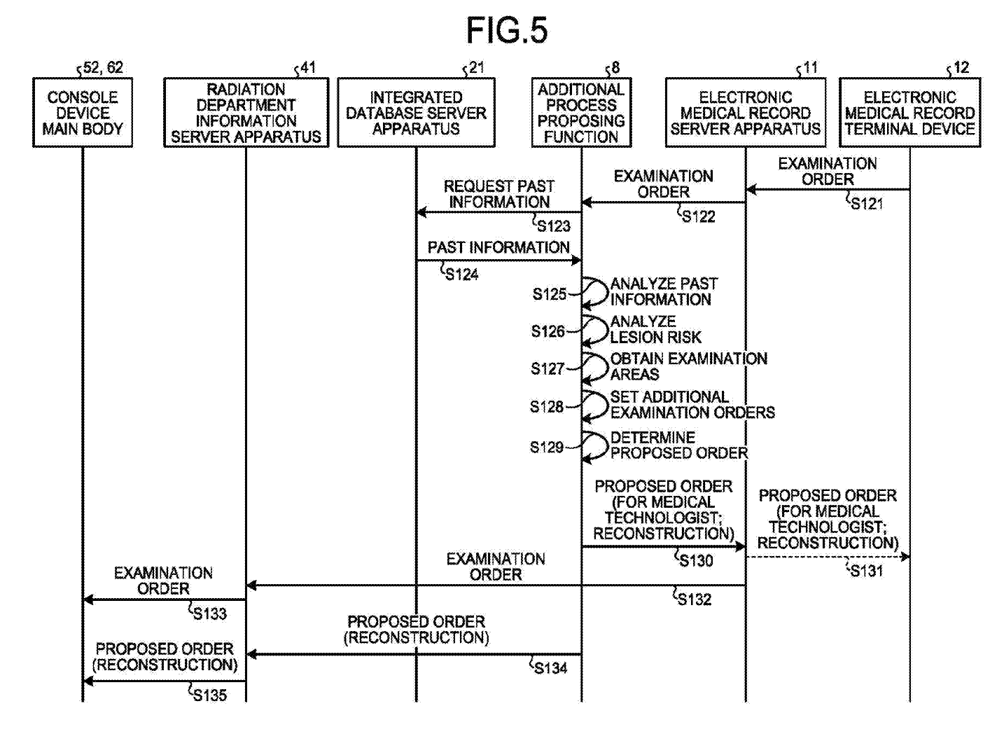

FIG. 3 is a sequence chart illustrating an example of a process performed when a proposed order suggesting expanding an examination area and an image taking area is presented to a doctor. In FIG. 3, let us assume that the doctor issued an examination order from the electronic medical record terminal device 12 to the electronic medical record server apparatus 11 (step S101). FIG. 4A is a drawing illustrating an example of the examination order. In FIG. 4, the examination order includes a doctor ID identifying the doctor, a patient ID identifying the patient, a site on which the examination is to be performed, and a modality to be used in the examination. In the present example, it is assumed that the site is the heart, whereas the modality is CT.

Returning to the description of FIG. 3, the electronic medical record server apparatus 11 informs the additional process proposing function 8 of details of the examination order (step S102). In this situation, instead of the details of the examination order being provided from the electronic medical record server apparatus 11 side for the additional process proposing function 8 side, another arrangement is also acceptable in which, from the additional process proposing function 8 side, it is checked to see whether a new examination order is present in the electronic medical record server apparatus 11, so as obtain details of the examination order when a new examination order is present. Further, while the electronic medical record system 1 is in an environment where direct communication with another system is not possible, the details of the examination order may be given to the additional process proposing function 8 through a human means.

When the examination order recognizing function 81 included in the additional process proposing function 8 recognizes the examination order (step S102), the past information obtaining function 82 requests past information from the integrated database server apparatus 21 by designating, for example, the patient ID included in the examination order (step S103). The integrated database server apparatus 21 checks to see whether or not the requested past information is present, and when the requested past information is present, responds with the past information (step S104). Examples of the obtained past information include non-image information such as a report related to a diagnosis made on the patient, as well as image information obtained through an examination performed on the patient or the like. Examples of the non-image information include a report or the like created by a doctor or a person in charge of an examination, as well as a result extracted by applying a segmentation process or a Computer-Aided Diagnosis (CAD) process to an image indicating a result of an examination. At the point in time when the past information is requested, it is possible to obtain, from the image serving as the past information, information appended to the image, as well as information extracted as a result of applying the segmentation process and/or the CAD process thereto in a real-time manner.

If the integrated database server apparatus 21 were not provided, the past information obtaining function 82 would request the past information from the electronic medical record server apparatus 11, the medical image management server apparatus 31, and the radiation department information server apparatus 41. When the past information is present in any other apparatus, the past information obtaining function 82 requests the past information from the apparatus. FIG. 4B is a drawing illustrating an example of the past information. In FIG. 4B, the past information includes the patient's ID, age, gender, and so on, as well as the patient's medical history, family medical histories, an imaging history, a blood test history, and the like. In the present example, it is assumed that the patient's medical history indicates liver cancer.

Returning to the description of FIG. 3, the past information analyzing function 831 included in the additional examination order setting function 83 analyzes the past information obtained by the past information obtaining function 82 (step S105). The past information analyzing function 831 extracts “liver cancer” in the patient's medical history, from the example of the past information in FIG. 4B.

Returning to the description of FIG. 3, on the basis of the past information analyzed by the past information analyzing function 831, the lesion risk analyzing function 832 speculates one or more lesions each having a possibility of occurring in the patient, as a lesion risk analysis (step S106). Next, an example of the lesion speculating process will be explained with reference to FIG. 4C. FIG. 4C is a drawing illustrating an example of a metastasis possibility judgment table. The metastasis possibility judgment table stores therein pieces of information about various types of tumors with which people can have previously been diagnosed and pieces of information of primary sites each having a possibility of metastasis, so as to be kept in correspondence with each other. For example, on the basis of “liver cancer” in the patient's medical history included in the past information in FIG. 4B, the lesion risk analyzing function 832 obtains “lungs” listed as the first item for “liver cancer” in the metastasis possibility judgment table illustrated in FIG. 4C and speculates a lesion of “lung cancer”. Further, on the basis of “liver cancer” in the patient's medical history, the lesion risk analyzing function 832 speculates a lesion of “liver cancer” that may be recurring. FIG. 4D is a drawing illustrating examples of lesion candidates. FIG. 4D illustrates the speculated lesion candidates “lung cancer” and “liver cancer”. In the present example, the lesion candidates are displayed together with reasons for the speculation; however, it is also acceptable to display the lesion candidates together with probability values or ranking indicating the possibility of occurrence for each of the lesions. The probability values and the raking may be configured, in advance, into the metastasis probability judgment table illustrated in FIG. 4C or may be set in advance for each of the lesions.

Returning to the description of FIG. 3, the examination area obtaining function 833 obtains examination areas corresponding to the lesions speculated by the lesion risk analyzing function 832 (step S107). For example, the examination area obtaining function 833 refers to a table or the like storing therein names of lesions and examination areas so as to be kept in correspondence with each other and obtains an examination area as a candidate, on the basis of the name of each of the lesions. For example, as the candidates for the examination areas, the examination area obtaining function 833 obtains “entire lung field region” on the basis of “lung cancer” and obtains “liver” on the basis of “liver cancer”. FIG. 4E is a drawing illustrating examples of the examination area candidates. FIG. 4E illustrates the obtained examination area candidates “site: entire lung field region” and “site: liver”.

Returning to the description of FIG. 3, the additional examination order extracting/setting function 834 sets one or more additional examination orders (candidates) from among the candidates obtained as the examination areas by the examination area obtaining function 833 (step S108). For example, while using the examination areas, the additional examination order extracting/setting function 834 sets the additional examination orders (candidates) in descending order of the possibility of occurrence of the lesions, on the basis of the reasons for the speculation or the like. FIG. 4F is a drawing illustrating examples of the additional examination orders (candidates). FIG. 4F illustrates “site: entire lung field region” and “site: liver” as the examples of the additional examination orders (candidates).

Returning to the description of FIG. 3, from among the additional examination orders, the proposed order determining function 84 determines a proposed order that is either to be presented to the user (e.g., a doctor, a medical technologist, or the like) or to be automatically executed, on the basis of a relationship between the examination order recognized by the examination order recognizing function 81 and the additional examination orders set by the additional examination order setting function 83 (step S109). To perform the process, the proposed order determining function 84 refers to a site map (a human body map) indicating inclusion relationships among examination areas. FIG. 4G is a drawing illustrating an example of the site map. For example, the proposed order determining function 84 compares “site: heart” in the examination order illustrated in FIG. 4A with “site: entire lung field region” and “site: liver” in the additional examination orders (candidates) illustrated in FIG. 4F and judges whether or not it is necessary to expand the examination area and the image taking area by referring to a site map such as that illustrated in FIG. 4G. The site map illustrated in FIG. 4G indicates only the inclusion relationships in the body axis direction in correspondence with an X-ray CT apparatus. In comparison to “site: heart” in the examination order, “site: entire lung field region” and “site: liver” in the additional examination orders (candidates) both require expansion of the examination area and the image taking area. Accordingly, because the radiation exposure amount increases, it is necessary to have a doctor make a judgment. FIG. 4H is a drawing illustrating an example of a proposed order. In FIG. 4H, “site: entire lung field region” and “site: heart+liver” to be presented to a doctor are determined. Alternatively, the proposed order determining function 84 may determine, as a proposed order, “site: entire lung field region+liver” by combining together “site: entire lung field region” and “site: heart+liver”.

Returning to the description of FIG. 3, the additional examination order proposing/executing function 85 notifies the electronic medical record server apparatus 11 of the proposed order suggesting expanding the examination area and the image taking area and having been determined by the proposed order determining function 84, so that the proposed order can be presented to the user (i.e., a doctor) (step S110). Further, while the electronic medical record system 1 is in an environment where direct communication with another system is not possible, the details of the proposed order may be given to the electronic medical record server apparatus 11 through a human means.

Subsequently, the electronic medical record server apparatus 11 notifies the electronic medical record terminal device 12 used by the doctor who issued the original examination order, of the proposed order suggesting expanding the examination area and the image taking area and having been provided in the notification from the additional process proposing function 8 (step S111). It is also acceptable to arrange a portable terminal device (e.g., a smartphone) carried by the doctor to be notified of the proposed order, separately from the electronic medical record terminal device 12.

The doctor checks the proposed order on the electronic medical record terminal device 12. When the doctor is to adopt the proposed order suggesting expanding the examination area and the image taking area, the doctor performs an examination order changing process on the electronic medical record server apparatus 11 (step S112). The electronic medical record server apparatus 11 notifies the radiation department information server apparatus 41 of the examination order after the change (step S113). The radiation department information server apparatus 41 notifies the console device main body 52 or 62 in the corresponding modality, of the examination order after the change (step S114). A medical technologist performs an examination according to the examination order of which the console device main body 52 or 62 was notified.

In the proposed order suggesting the expansion of the examination area and the image taking area, it is assumed that each of the areas is expanded while being centered on the original area, within the range of the modality (e.g., CT) designated in the original examination order. However, the proposed order may suggest expansion (addition) of an area that is noncontiguous or may further suggest that the modality should be changed.

Step S102 illustrated in FIG. 3 is a step corresponding to the examination order recognizing function 81. Step S102 is a step at which the examination order recognizing function 81 is realized as a result of the processing circuit 411 reading and executing a program corresponding to the examination order recognizing function 81 from the storage circuit 414.

Steps S103 and S104 illustrated in FIG. 3 are steps corresponding to the past information obtaining function 82. Steps S103 and S104 are steps at which the past information obtaining function 82 is realized as a result of the processing circuit 411 reading and executing a program corresponding to the past information obtaining function 82 from the storage circuit 414.

Step S105 illustrated in FIG. 3 is a step corresponding to the past information analyzing function 831 included in the additional examination order setting function 83. Step S105 is a step at which the past information analyzing function 831 is realized as a result of the processing circuit 411 reading and executing a program corresponding to the past information analyzing function 831 from the storage circuit 414.

Step S106 illustrated in FIG. 3 is a step corresponding to the lesion risk analyzing function 832 included in the additional examination order setting function 83. Step S106 is a step at which the lesion risk analyzing function 832 is realized as a result of the processing circuit 411 reading and executing a program corresponding to the lesion risk analyzing function 832 from the storage circuit 414.

Step S107 illustrated in FIG. 3 is a step corresponding to the examination area obtaining function 833 included in the additional examination order setting function 83. Step S107 is a step at which the examination area obtaining function 833 is realized as a result of the processing circuit 411 reading and executing a program corresponding to the examination area obtaining function 833 from the storage circuit 414.

Step S108 illustrated in FIG. 3 is a step corresponding to the additional examination order extracting/setting function 834 included in the additional examination order setting function 83. Step S108 is a step at which the additional examination order extracting/setting function 834 is realized as a result of the processing circuit 411 reading and executing a program corresponding to the additional examination order extracting/setting function 834 from the storage circuit 414.

Step S109 illustrated in FIG. 3 is a step corresponding to the proposed order determining function 84. Step S109 is a step at which the proposed order determining function 84 is realized as a result of the processing circuit 411 reading and executing a program corresponding to the proposed order determining function 84 from the storage circuit 414.

Step S110 illustrated in FIG. 3 is a step corresponding to the additional examination order proposing/executing function 85. Step S110 is a step at which the additional examination order proposing/executing function 85 is realized as a result of the processing circuit 411 reading and executing a program corresponding to the additional examination order proposing/executing function 85 from the storage circuit 414.

FIG. 5 is a sequence chart illustrating an example of a process performed when a proposed order suggesting a reconstruction process is presented to a medical technologist. In FIG. 5, it is assumed that a medical order issued an examination order from the electronic medical record terminal device 12 to the electronic medical record server apparatus 11 (step S121). An example of the examination order is the same as the one illustrated in FIG. 4A. In other words, the site is the heart, while the modality is CT.

Returning to the description of FIG. 5, the electronic medical record server apparatus 11 notifies the additional process proposing function 8 of details of the examination order (step S122). As explained above, the other arrangements are also similarly acceptable in which, from the additional process proposing function 8 side, it is checked to see whether a new examination order is present in the electronic medical record server apparatus 11 and in which the details of the examination order are given to the additional process proposing function 8 through a human means.

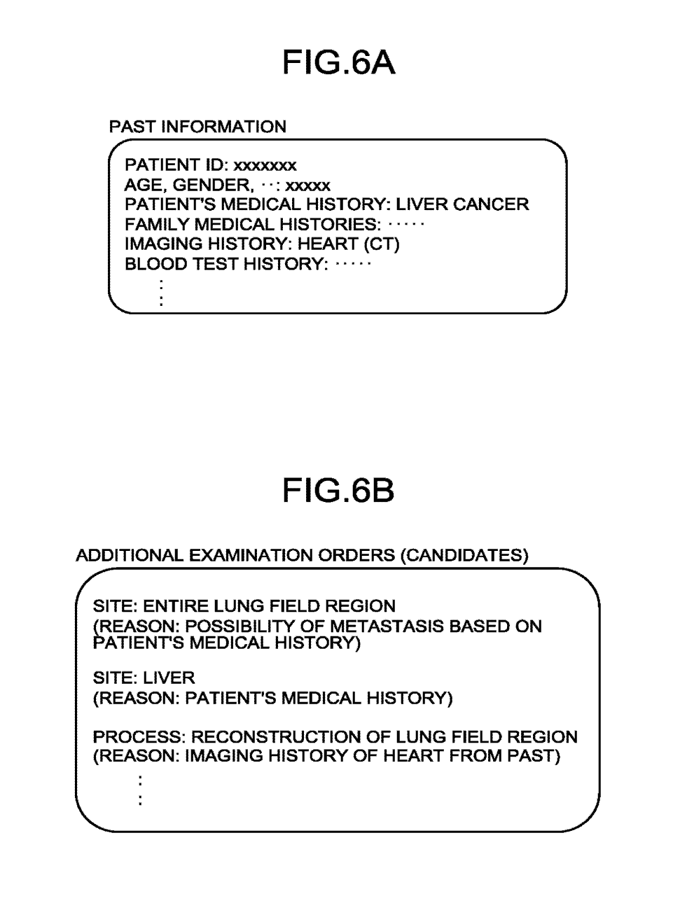

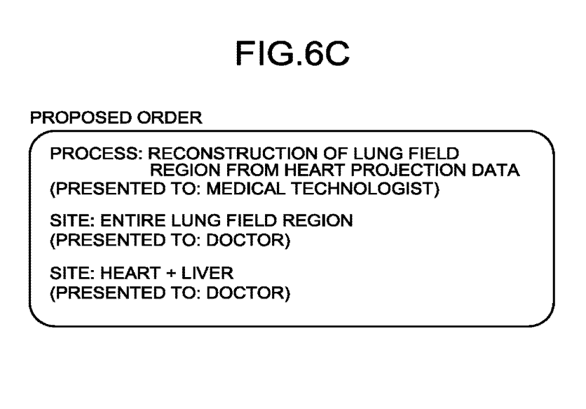

When the examination order recognizing function 81 included in the additional process proposing function 8 recognizes the examination order (step S122), the past information obtaining function 82 requests past information from the integrated database server apparatus 21 by designating, for example, the patient ID (step S123). The integrated database server apparatus 21 checks to see whether or not the requested past information is present, and when the requested past information is present, responds with the past information (step S124). If the integrated database server apparatus 21 were not provided, the past information obtaining function 82 would request the past information from the electronic medical record server apparatus 11, the medical image management server apparatus 31, and the radiation department information server apparatus 41. When the past information is present in any other apparatus, the past information obtaining function 82 requests the past information from the apparatus. FIG. 6A is a drawing illustrating an example of the past information. In FIG. 6A, the past information includes the patient's ID, age, gender, and so on, as well as the patient's medical history, family medical histories, an imaging history, a blood test history, and the like. In the present example, it is assumed that the patient's medical history indicates liver cancer and that the imaging history indicates “heart (CT)” (the heart was imaged by using an X-ray CT apparatus).

Returning to the description of FIG. 5, the past information analyzing function 831 included in the additional examination order setting function 83 analyzes the past information obtained by the past information obtaining function 82 (step S125). The past information analyzing function 831 extracts “liver cancer” in the patient's medical history and “heart (CT)” in the imaging history, from the example of the past information in FIG. 6A.

Returning to the description of FIG. 5, on the basis of the past information analyzed by the past information analyzing function 831, the lesion risk analyzing function 832 speculates one or more lesions each having a possibility of occurring in the patient, as a lesion risk analysis (step S126). For example, on the basis of “liver cancer” in the patient's medical history, the lesion risk analyzing function 832 speculates “lungs”, i.e., a lesion of “lung cancer”, from a metastasis possibility judgment table such as that illustrated in FIG. 4C. Further, on the basis of “liver cancer” in the patient's medical history, the lesion risk analyzing function 832 speculates a lesion of “liver cancer” that may be recurring. The speculated lesion candidates are the same as those illustrated in FIG. 4D. In the present example, the lesion candidates are displayed together with the reasons for the speculation; however, it is also acceptable to display the lesion candidates together with probability values or ranking indicating the possibility of occurrence for each of the lesions.

Returning to the description of FIG. 5, the examination area obtaining function 833 obtains examination areas corresponding to the lesions speculated by the lesion risk analyzing function 832 (step S127). For example, the examination area obtaining function 833 obtains “entire lung field region” from “lung cancer” and obtains “liver” from “liver cancer”, as candidates for the examination areas. Examples of the obtained examination area candidates are the same as those illustrated in FIG. 4E.

Returning to the description of FIG. 5, the additional examination order extracting/setting function 834 sets one or more additional examination orders (candidates) from among the candidates obtained as the examination areas by the examination area obtaining function 833 (step S128). In this situation, while using the examination areas, the additional examination order extracting/setting function 834 sets the additional examination orders (candidates) in descending order of the possibility of occurrence, on the basis of the reasons for the speculation or the like, and further judges whether or not it is possible to substitute all or a part of the examination areas with a reconstructed image of either a part within an image taking area used in the past indicated in the image taking history or a part that is within a new image taking area but is not subject to an examination. When the substitution is possible, the additional examination order extracting/setting function 834 sets an additional examination order (candidate) indicating the reconstruction process. For example, with respect to each of the items such as “site: entire lung field region” and “site: liver” illustrated as the examination area candidates in FIG. 4E, the additional examination order extracting/setting function 834 compares the relationship thereof with “imaging history: heart (CT)” in the past information by referring to the site map in FIG. 4G and determines that it is possible to reconstruct a part of the “lung field region” from raw data of “heart (CT)” performed in the past. The additional examination order extracting/setting function 834 thus sets the reconstruction process in an additional examination order (a candidate). In other words, in the imaging process performed on the “heart (CT)” in the past, the imaging process must have been performed also on the lung field region which overlaps the heart in the body axis direction, and the raw data must be present. However, because no reconstruction process was performed on the lung field region, the raw data of the lung field region was not used for the diagnosis. Accordingly, it is possible to substitute a part of the examination areas by performing a reconstruction process on the raw data with respect to the lung field region. The reconstruction process in this situation is not an examination of the entire “lungs”, but is able to provide useful information for checking the state of the “lungs” without increasing the radiation exposure amount. FIG. 6B is a drawing illustrating examples of the additional examination orders (candidates). In FIG. 6B, as the examples of the additional examination orders (candidates), “process: reconstruction of lung field region” is added to “site: entire lung field region” and “site: liver”.

Returning to the description of FIG. 5, from among the additional examination orders, the proposed order determining function 84 determines a proposed order that is either to be presented to the user (e.g., a doctor, a medical technologist, or the like) or to be automatically executed, on the basis of a relationship between the examination order recognized by the examination order recognizing function 81 and the additional examination orders set by the additional examination order setting function 83 (step 129). For example, the proposed order determining function 84 compares “site: heart” in the examination order illustrated in FIG. 4A with “site: entire lung field region” and “site: liver” in the additional examination orders (candidates) illustrated in FIG. 6B and judges whether or not it is necessary to expand the examination area and the image taking area by referring to a site map (a human body map) indicating inclusion relationships among examination areas such as that illustrated in FIG. 4G. In comparison to “site: heart” in the examination order, “site: entire lung field region” and “site: liver” in the additional examination orders (candidates) both require expansion of the examination area and the image taking area. Accordingly, because the radiation exposure amount increases, it is necessary to have a doctor make a judgment. Thus, “site: entire lung field region” and “site: heart+liver” to be presented to a doctor are determined.

Further, as for “process: reconstruction of lung field region” in one of the additional examination orders (candidates), it is not necessary to perform a new imaging process, and it is sufficient to only perform the process on the existing raw data. Accordingly, because it is not requisite to check with doctors, this additional examination order will be presented to a medical technologist. However, because the result of the reconstruction process shall be used for diagnosis purposes, it is meaningful to provide the doctor who issued the original examination order with the information. Further, in some hospitals, doctors also serve as medical technologists, and it may not be necessary to distinguish doctors from medical technologists. Further, it is also acceptable to limit the situations where a proposed order contains details of a reconstruction process to only when the additional process proposing function 8 is unable to automatically execute the reconstruction process (for example, being unable to request the reconstruction process due to an issue related to access right to the raw data or processing software). Further, the additional process proposing function 8 may be configured not to automatically control the execution of the reconstruction process so that the proposed order is always presented to a medical technologist.

FIG. 6C is a drawing illustrating an example of the proposed order. FIG. 6C illustrates the example of the proposed order includin “process: reconstruction of lung field region from heart projection data” to be presented to a medical technologist and “site: entire lung field region” and “site: heart+liver” to be presented to a doctor. Alternatively, the proposed order may include “site: entire lung field region+liver” in which “site: entire lung field region” and “site: heart+liver” are combined together. Further, when proposed orders include a proposed order concerning a site or a region that is duplicate between a doctor and a medical technologist, it is desirable to issue an alert when the proposed orders are presented, because when one of the duplicate proposed orders is adopted, the other proposed order may no longer be necessary.

The example is explained above in which the reconstruction process from the past raw data is proposed on the basis of the imaging history of the patient in the past information; however, another arrangement is also acceptable in which a reconstruction process is proposed from within an image taking area on which an examination is going to be performed on the basis of the examination order issued by the doctor. For example, when “site: heart” and “modality: CT” were designated in the original examination order, and a metastasis to “lung cancer” from “liver cancer” in the patient's medical history is speculated, it is possible to propose a reconstruction process of a “lungs” section that is to be imaged at the same time by implementing an X-ray CT procedure, while keeping the “heart” as the examined site, instead of expanding the examination area to the “entire lung field region”.

When the proposed order determined by the proposed order determining function 84 contains only one or more reconstruction processes, the processes at steps S130 through S135 in FIG. 5 shall be performed. When the proposed order determined by the proposed order determining function 84 contains both expansion of the examination area and the image taking area and one or more reconstruction processes, a process obtained by combining together the processes at steps S110 through S114 in FIG. 3 and the processes at steps S130 through S135 in FIG. 5 shall be performed.

In FIG. 5, the additional examination order proposing/executing function 85 notifies the electronic medical record server apparatus 11 of the proposed order suggesting the reconstruction process, so that the proposed order can be presented to the user (i.e., a medical technologist) (step S130). While the electronic medical record system 1 is in an environment where direct communication with another system is not possible, the details of the proposed order may be given to the electronic medical record server apparatus 11 through a human means.

Subsequently, although the procedure is not requisite, the electronic medical record server apparatus 11 notifies the electroni medical record terminal device 12 used by the doctor or the like who issued the original examination order of the proposed order provided in the notification from the additional process proposing function 8, as reference information (step S131).

Further, the electronic medical record server apparatus 11 notifies the radiation department information server apparatus 41 of the original examination order issued by the doctor (step S132). The radiation department information server apparatus 41 notifies the console device main body 52 (or 62) in the corresponding modality, of the examination order provided in the notification (step S133).

Further, the additional examination order proposing/executing function 85 included in the additional process proposing function 8 notifies the radiation department information server apparatus 41 of the proposed order suggesting the reconstruction process to be presented to a medical technologist (step S134). The radiation department information server apparatus 41 notifies the console device main body 52 (or 62) in the corresponding modality, of the proposed order indicating the reconstruction process and having been provided in the notification (step S135). The medical technologist who has checked the examination order and the proposed order performs the examination according to the examination order, and further, when adopting the proposed order, the medical technologist performs the reconstruction process according to the proposed order. It is also acceptable to arrange a portable terminal device (e.g., a smartphone) carried by the medical technologist to be notified of the examination order and the proposed order, separately from the console device main body 52 (or 62).

In the above example, the proposed order suggesting the reconstruction process is assumed to be in the same modality as the modality (e.g., CT) designated in the original examination order. However, it is also acceptable when the proposed order suggests a modality different from that in the original examination order.

When the proposed order contains both expansion of the image taking area and one or more reconstruction processes, the process at step S114 in FIG. 3 shall be followed by the processes at steps S130, S131, S134, and S135 illustrated in FIG. 5.

Step S122 illustrated in FIG. 5 is a step corresponding to the examination order recognizing function 81. Step S122 is a step at which the examination order recognizing function 81 is realized as a result of the processing circuit 411 reading and executing a program corresponding to the examination order recognizing function 81 from the storage circuit 414.

Steps S123 and S124 illustrated in FIG. 5 are steps corresponding to the past information obtaining function 82. Steps S123 and S124 are steps at which the past information obtaining function 82 is realized as a result of the processing circuit 411 reading and executing a program corresponding to the past information obtaining function 82 from the storage circuit 414.

Step S125 illustrated in FIG. 5 is a step corresponding to the past information analyzing function 831 included in the additional examination order setting function 83. Step S125 is a step at which the past information analyzing function 831 is realized as a result of the processing circuit 411 reading and executing a program corresponding to the past information analyzing function 831 from the storage circuit 414.

Step S126 illustrated in FIG. 5 is a step corresponding to the lesion risk analyzing function 832 included in the additional examination order setting function 83. Step S126 is a step at which the lesion risk analyzing function 832 is realized as a result of the processing circuit 411 reading and executing a program corresponding to the lesion risk analyzing function 832 from the storage circuit 414.

Step S127 illustrated in FIG. 5 is a step corresponding to the examination area obtaining function 833 included in the additional examination order setting function 83. Step S127 is a step at which the examination area obtaining function 833 is realized as a result of the processing circuit 411 reading and executing a program corresponding to the examination area obtaining function 833 from the storage circuit 414.

Step S128 illustrated in FIG. 5 is a step corresponding to the additional examination order extracting/setting function 834 included in the additional examination order setting function 83. Step S123 is a step at which the additional examination order extracting/setting function 834 is realized as a result of the processing circuit 411 reading and executing a program corresponding to the additional examination order extracting/setting function 834 from the storage circuit 414. Step S129 illustrated in FIG. 5 is a step corresponding to the proposed order determining function 84. Step S129 is a step at which the proposed order determining function 84 is realized as a result of the processing circuit 411 reading and executing a program corresponding to the proposed order determining function 84 from the storage circuit 414.

Steps S130 and S134 illustrated in FIG. 5 are steps corresponding to the additional examination order proposing/executing function 85. Steps S130 and S134 are steps at which the additional examination order proposing/executing function 85 is realized as a result of the processing circuit 411 reading and executing a program corresponding to the additional examination order proposing executing function 85 from the storage circuit 414.

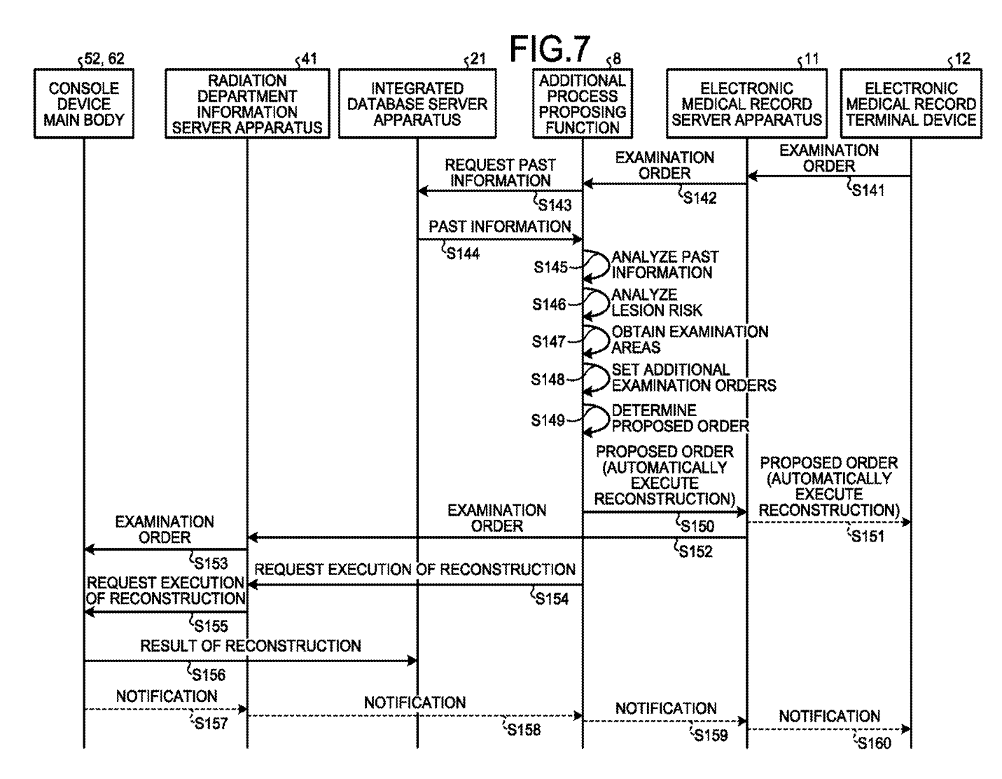

Next, FIG. 7 is a sequence chart illustrating an example of a process performed when the reconstruction process is automatically performed. In FIG. 7, the processes at steps S141 through S148 are the same as the processes at step S121 through S128 illustrated in FIG. 5.

In the example in FIG. 7, when determining, from among the additional examination orders, the proposed order that is either to be presented to a user (e.g., a doctor, a medical technologist, or the like) or to be automatically executed, (step S149), the proposed order determining function 84 determines that the proposed order suggesting a reconstruction process is to be automatically executed. For example, when there is no issue related to access right to the raw data or processing software or the like, so that it is possible to automatically control the execution of the reconstruction process, the additional process proposing function 8 determines that the proposal order suggesting the reconstruction process is to be executed automatically.

As a subsequent process, when the proposed order determined by the proposed order determining function 84 contains only one or more reconstruction processes, the processes at steps S150 through S160 in FIG. 7 shall be performed. When the proposed order determined by the proposed order determining function 84 contains both expansion of the examination area and the image taking area and one or more reconstruction processes, a process obtained by combining together the processes at steps S110 through S114 illustrated in FIG. 3 and the processes at steps S150 through S160 illustrated in FIG. 7 shall be performed.

In FIG. 7, the additional examination order proposing/executing function 85 notifies the electronic medical record server apparatus 11 that the proposed order suggesting the reconstruction process will automatically be executed (step S150). In this situation, while the electronic medical record system 1 is in an environment where direct communication with another system is not possible, the details of the proposed order may be given to the electronic medical record server apparatus 11 through a human means.

Subsequently, although the procedure is not requisite, the electronic medical record server apparatus 11 notifies the electronic medical record terminal device 12 used by the doctor or the like who issued the original examination order of the proposed order provided in the notification from the additional process proposing function 8, as reference information (step S151).

Further, the electronic medical record server apparatus 11 notifies the radiation department information server apparatus 41 of the original examination order issued by the doctor (step S152). The radiation department information server apparatus 41 notifies the console device main body 52 (or 62) in the corresponding modality, of the examination order provided in the notification (step S153).

Further, the additional examination order proposing/executing function 85 included in the additional process proposing function 8 notifies the radiation department information server apparatus 41 of the request to execute the reconstruction process based on the proposed order suggesting the reconstruction process (step S154). The radiation department information server apparatus 41 notifies the console device main body 52 (or 62) in the corresponding modality, of the reconstruction execution request provided in the notification (step S155). The reconstruction execution request includes information used for identifying the raw data from the imaging process performed in the past. When the software (a computer program) used for the reconstruction process is present in a device other than the console device main body 52 (or 62), it is also acceptable to request the device to execute the reconstruction process.

When having received the execution request, the console device main body 52 (or 62) executes the reconstruction process, and when the reconstruction process has been executed successfully, the console device main body 52 (or 62) stores an image resulting from the reconstruction process into the integrated database server apparatus 21 (step S156). When the reconstruction process has been performed successfully, it is desirable to further arrange a segmentation process and/or a CAD process to also be automatically performed on the resulting image, so that results thereof can be stored into the integrated database server apparatus 21. The results of the segmentation process and/or the CAD process serve as information useful for diagnosis processes performed by doctors, and the like.

Although the procedure is not requisite, the console device main body 52 (or 62) notifies the radiation department information server apparatus 41 that the reconstruction process has been performed (step S157). The radiation department information server apparatus 41 notifies the additional process proposing function B of the same (step S158). The additional examination order proposing/executing function 85 included in the additional process proposing function 8 notifies the electronic medical record server apparatus 11 that the reconstruction process has been performed (step S159). The electronic medical record server apparatus 11 notifies the electronic medical record terminal device 12 used by the doctor of the same (step S160). The doctor refers to the result of the reconstruction process as necessary and uses the information for diagnoses and treatments for the patient.

Step S142 illustrated in FIG. 7 is a step corresponding to the examination order recognizing function 81. Step S142 is a step at which the examination order recognizing function 81 is realized as a result of the processing circuit 411 reading and executing a program corresponding to the examination order recognizing function 81 from the storage circuit 414.

Steps S143 and S144 illustrated in FIG. 7 are steps corresponding to the past information obtaining function 82. Steps S143 and S144 are steps at which the past information obtaining function 82 is realized as a result of the processing circuit 411 reading and executing a program corresponding to the past information obtaining function 82 from the storage circuit 414.

Step S145 illustrated in FIG. 7 is a step corresponding to the past information analyzing function 831 included in the additional examination order setting function 83. Step S145 is a step at which the past information analyzing function 831 is realized as a result of the processing circuit 411 reading and executing a program corresponding to the past information analyzing function 831 from the storage circuit 414.

Step S146 illustrated in FIG. 7 is a step corresponding to the lesion risk analyzing function 832 included in the additional examination order setting function 83. Step S146 is a step at which the lesion risk analyzing function 832 is realized as a result of the processing circuit 411 reading and executing a program corresponding to the lesion risk analyzing function 832 from the storage circuit 414.

Step S147 illustrated in FIG. 7 is a step corresponding to the examination area obtaining function 833 included in the additional examination order setting function 83. Step S147 is a step at which the examination area obtaining function 833 is realized as a result of the processing circuit 411 reading and executing a program corresponding to the examination area obtaining function 833 from the storage circuit 414.

Step S148 illustrated in FIG. 7 is a step corresponding to the additional examination order extracting/setting function 834 included in the additional examination order setting function 83. Step S148 is a step at which the additional examination order extracting/setting function 834 is realized as a result of the processing circuit 411 reading and executing a program corresponding to the additional examination order extracting/setting function 834 from the storage circuit 414.

Step S149 illustrated in FIG. 7 is a step corresponding to the proposed order determining function 84. Step S149 is a step at which the proposed order determining function 84 is realized as a result of the processing circuit 411 reading and executing a program corresponding to the proposed order determining function 84 from the storage circuit 414.

Steps S150, S154, and S159 illustrated in FIG. 7 are steps corresponding to the additional examination order proposing/executing function 85. Steps S150, S154, and S159 are steps at which the additional examination order proposing/executing function 85 is realized as a result of the processing circuit 411 reading and executing a program corresponding to the additional examination order proposing/executing function 85 from the storage circuit 414.

As explained above, according to the first embodiment, with respect to the examination order issued by the doctor, the proposal is made to suggest the expansion of the examination area and the image taking area or the reconstruction process, on the basis of the past information related to the patient. In addition, the reconstruction process is executed automatically when possible. Accordingly, it is possible to assist the user in discovering a lesion in a non region of interest. Further, the first embodiment is also expected to be utilized as supplementary data when development is observed (as a follow-up) after a treatment applied to the patient in the past.

### Second Embodiment

In a second embodiment, additional costs are evaluated with respect to additional examination orders or a proposed order, so as to append the evaluated additional costs as reference information to a presented proposed order or so as to use the evaluated additional costs as an index for determining a proposed order from among the additional examination orders. Examples of the additional costs include an increase in the radiation exposure amount due to expansion of the image taking area, an increase in the acquisition time period due to an enlargement of the image taking area, an increase in the processing time period required by the reconstruction process, and the like. Because the proposed order will be presented together with the additional costs, it becomes easier for the doctor or the medical technologist to determine whether or not the proposed order should be adopted. In other words, regarding the adoption of the proposed order, it becomes easier for the doctor or the medical technologist to determine whether or not the proposed order should be adopted, by considering whether or not it is worth bearing the presented additional costs. Further, because a proposed order may automatically be determined from among the additional examination orders on the basis of the additional costs, the classification among the destinations to whom the proposed order is to be presented becomes clearer. For example, when there is an increase in the radiation exposure amount, it is understood there is a high possibility that the image taking area may be enlarged. It is therefore more clearly indicated that the proposed order should be presented to a doctor. An increase in the acquisition time period can also be handled in the same manner as an increase in the radiation exposure amount. Further, even when there is an increase in the processing time period required by a construction process, unless there is an increase in the radiation exposure amount, there is no need to present the proposed order to a doctor. It is therefore more clearly indicated that it is possible to present the proposed order to a medical technologist or to have the proposed order executed automatically.

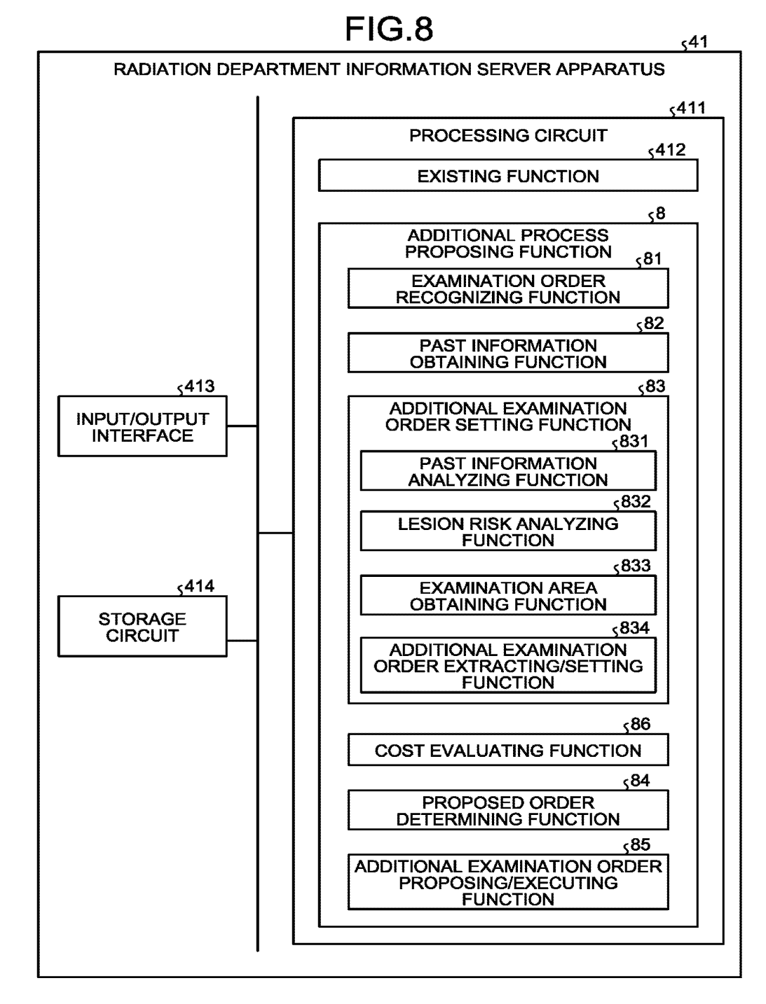

FIG. 8 is a diagram illustrating an exemplary configuration in which the radiation department information server apparatus 41 is provided with the additional process proposing function 8 according to the second embodiment. The configuration illustrated in FIG. 8 is almost the same as the configuration illustrated in FIG. 2, except that the additional process proposing function 8 is further provided with a cost evaluating function 86. The cost evaluating function 86 is configured to evaluate additional costs of the additional examination orders (candidates) set by the additional examination order setting function 83 and to append the evaluated additional costs to the additional examination orders (candidates) as reference information.

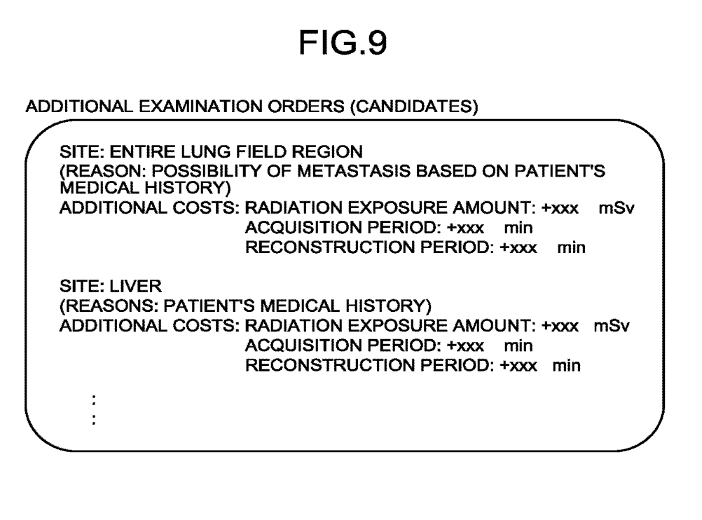

As for the details of the process, the descriptions of the process explained with reference to FIGS. 3, 5, and 7 are slightly changed. More specifically, a processing step of evaluating the additional costs performed by the cost evaluating function 86 are added immediately after step S108 in FIG. 3, step S128 in FIG. 5, and step S148 in FIG. 7. At the processing step of evaluating the additional costs performed by the cost evaluating function 86, calculations are performed to obtain an increase in the radiation exposure amount due to the expansion of the image taking area, an increase in the acquisition time period due to an enlargement of the image taking area, an increase in the processing time period required by the reconstruction process, and/or the like in accordance with details of the additional examination orders (candidates), so that information about the additional costs can be appended to the additional examination orders (candidates). FIG. 9 is a drawing illustrating examples of additional examination orders (candidates). In FIG. 9, for each of the items such as “site: entire lung field region” and “site: liver” indicated in the additional examination orders (candidates), a radiation exposure amount, an acquisition time period, and a reconstruction time period are calculated and appended thereto.

Further, in the proposed order determining process performed by the proposed order determining function 84 at step S109 in FIG. 3, step S129 in FIG. 5, and step S149 in FIG. 7, any of the proposed order having an additional cost with an increase in the radiation exposure amount is classified a a proposed order to be presented to a doctor, whereas any of the proposed orders having an additional cost with no increase in the radiation exposure amount is classified as a proposed order to be presented to a medical technologist. In other words, it is possible to classify the proposed orders according to the radiation exposure amounts calculated as the additional costs and to indicate the processes clearer, without the need to distinguish whether or not each of the proposed orders involves a reconstruction process or not.

Further, when the proposed order is presented to a doctor or a medical technologist at steps S110 and S111 in FIG. 3, at steps S130, S131, S134, and S135 in FIG. 5, and at steps S150 and S151 in FIG. 7, the additional costs evaluated by the cost evaluating function 86 are additionally presented. In this situation, an arbitrary format may be used for presenting the additional costs. For example, the details of the additional costs may be presented together with the proposed order displayed as a list.

When the proposed order is presented to the doctor or the medical technologist by the additional examination order proposing/executing function 85 as described above, it is also acceptable to present diagnosis resource materials (e.g., examination results, acquired images, and the like) of the examined subject (the patient) from the past together therewith. In that situation, the past information obtaining function 82 included in the additional process proposing function 8 obtains non-image and/or image relevant information as the past information, on the basis of the patient ID or the like and forwards the obtained information to the additional examination order proposing/executing function 85. Examples of the images obtained in this situation include, not only the images stored in the integrated database server apparatus 21 or the like, but also images that are, separately from the proposed order explained above, reconstructed from raw data that was acquired in a past examination and stored in the integrated database server apparatus 21 or the like. The reconstruction process realized by using the raw data may be performed in a real-time manner or may be performed in the background at an appropriate time.

To obtain the relevant information, a machine learning scheme may be used. For example, it is acceptable to implement a machine learning process by using a relationship between the state of patients and pieces of information referenced to make diagnoses as a corpus, on the basis of a large number of diagnosis examples from the past. Accordingly, the past information obtaining function 82 is able to obtain, with a higher priority, certain types of information and details thereof speculated by the machine learning process on the basis of a current state of a patient. When presenting the proposed order to the doctor or the medical technologist, the additional examination order proposing/executing function 85 presents the non-image and/or image relevant information together therewith. In that situation, an arbitrary format may be used for presenting the relevant information. For example, details of the relevant information may directly be incorporated in the notification. Alternatively, a link to a reference address may be attached thereto.

The processing step of evaluating the additional costs described above is a step corresponding to the cost evaluating function 86. The processing step of evaluating the additional costs is a step at which the cost evaluating function 86 is realized as a result of the processing circuit 411 reading and executing a program corresponding to the cost evaluating function 86 from the storage circuit 414.

According to at least one aspect of the embodiments described above, it is possible to assist the user in discovering a lesion in the non region of interest.

While certain embodiments have been described, these embodiments have been presented by way of example only, and are not intended to limit the scope of the inventions. Indeed, the novel embodiments described herein may be embodied in a variety of other forms; furthermore, various omissions, substitutions and changes in the form of the embodiments described herein may be made without departing from the spirit of the inventions. The accompanying claims and their equivalents are intended to cover such forms or modifications as would fall within the scope and spirit of the inventions.

What is claimed is:

1. A hospital information apparatus comprising a processing circuit configured:
to obtain past information related to a subject for whom an examination order is issued;
to set one or more additional examination orders on a basis of an analysis performed on the past information; and
to determine a proposed order from among the additional examination orders on a basis of a relationship between the examination order and the additional examination orders.

2. The hospital information apparatus according to claim 1, wherein the additional examination orders are for examining the subject for a lesion suspected to occur in the subject.

3. The hospital information apparatus according to claim 1, wherein the proposed order is an examination order that is either to be presented to a user or to be executed.

4. The hospital information apparatus according to claim 1, wherein the additional examination orders are for examining the subject for a lesion that is different from a lesion for which an examination is performed under the examination order.

5. The hospital information apparatus according to claim 1, wherein the examination order and the additional examination orders each instruct either that an image of the subject be taken by using a medical image diagnosis apparatus or that a specimen acquired from the subject be analyzed.

6. The hospital information apparatus according to claim 1, wherein
the processing circuit is configured to speculate a lesion suspected to occur in the subject on a basis of the past information, and
the processing circuit sets the additional examination orders for examining the subject for the speculated lesion.

7. The hospital information apparatus according to claim 1, wherein the processing circuit determines at least one of the additional examination orders indicating that an image taking area of the examination order be expanded, as the proposed order to be presented to a user.

8. The hospital information apparatus according to claim 1, wherein the processing circuit determines at least one of the additional examination orders indicating that a reconstruction process be performed by using one selected from between data from past and data newly imaged, as the proposed order to be presented to a user.

9. The hospital information apparatus according to claim 1, wherein, when it is possible to execute, without a user's confirmation, at least one of the additional examination orders indicating that a reconstruction process be performed by using one selected from between data from past and data newly imaged, the processing circuit determines said at least one of the additional examination orders as the proposed order to be executed automatically.

10. The hospital information apparatus according to claim 1, wherein
the processing circuit is configured to calculate an additional cost of executing each of the additional examination orders, and
the processing circuit determines the proposed order on a basis of the additional costs.

11. The hospital information apparatus according to claim 10, wherein, when the proposed order is presented, the additional costs are also presented together therewith.

12. The hospital information apparatus according to claim 1, wherein, when the proposed order is presented, image or non-image information related to the proposed order is presented together therewith.

13. A hospital information system comprising:
an obtaining circuit configured to obtain past information related to a subject for whom an examination order is issued;
a setting circuit configured to set one or more additional examination orders on a basis f an analysis performed on the past information; and
a determining circuit configured to determine a proposed order from among the additional examination orders on a basic of a relationship between the examination order and the additional examination orders.

14. A hospital information processing method comprising:
obtaining past information related to a subject for whom an examination order is issued;
setting one or more additional examination orders on a basis of an analysis performed on the past information; and
determining a proposed order from among the additional examination orders on a basis of a relationship between the examination order and the additional examination orders.
